(19) United States
(12) Patent Application Publication
(10) Pub. No.: US 2019/0008798 A1
(43) Pub. Date: Jan. 10, 2019

(54) Coenzyme Q10 in the preventive treatment of type 2 diabetes

(71) Applicant: Pharma Nord ApS, Vojens (DK)

(72) Inventors: Sven MOESGAARD, Almind (DK); Urban Alehagen, Linköping (SE)

(21) Appl. No.: 16/005,949

(22) Filed: Jun. 12, 2018

(30) Foreign Application Priority Data
Jul. 5, 2017 (EP) 17179797.0

(51) Int. Cl. A61K 31/122; A61K 33/04; A61P 3/10

(52) U.S. Cl. CPC A61K 31/122; A61P 3/10; A61K 33/04

(57) ABSTRACT

The present disclosure relates to Q10 for use in the treatment or prophylactic treatment of type 2 diabetes in a human subject, said human subject being selected from the group consisting of; i) humans with decreased insulin sensitivity (increased HOMA-IR), ii) humans with low IGFBP-1 and adiponectin fasting levels, iii) humans above 40 years of age (with a family history of diabetes), iv) women with a history of gestational diabetes, and v) subjects with the metabolic syndrome (hypertension, abdominal obesity, high triglycerides but not T2DM).

### STATEMENT REGARDING PRIOR DISCLOSURES BY THE INVENTOR OR A JOINT INVENTOR (37 C.F.R. § 1.77(b)(6)

Research article entitled “Increase in insulin-like growth factor 1 (IGF-1) and insulin-like growth factor binding protein 1 after supplementation with selenium and coenzyme Q10. A prospective randomized double-blind placebo-controlled trial among elderly Swedish citizens”; PLOS ONE; XP-002775983; https://doi.org/10.1371/journal.pone.0178614; dated Jun. 13, 2017. An author of this research article is Urban Alehagen, a named co-inventor of the instant application.

### FIELD

The aspects of the embodiments disclosed relate to Coenzyme Q10 for use in the treatment or prophylactic treatment of a human subject, said treatment comprising the use of Coenzyme Q10 for preventing a decrease in serum levels of IGF-1.

The present disclosure particularly relates to Coenzyme Q10 for use in the prophylactic treatment of type 2 diabetes in a human subject.

Even more particularly, the present disclosure relates to Coenzyme Q10 for use in the prophylactic treatment of type 2 diabetes in a human subject, said treatment comprising daily administration of 100 mg Coenzyme Q10 or more for at least 3 months.

### BACKGROUND

The number of people suffering from diabetes type 2 (T2DM) is increasing in all countries of the world. It has been estimated that 10% of the population will suffer from diabetes within the year 2027, obesity being the driving force behind this escalating diabetes epidemic. Visceral/abdominal fat, i.e. increased waist circumference, is associated with the development of T2DM due primarily to an increase in insulin production, insulin resistance and activity of insulin-like growth factor I (IGF-I). Diabetes may lead to tissue damage causing renal failure, nerve dysfunction, cardiovascular disease (CVD), reduced vision, chronic foot ulcers, amputation, depression, arthrosis and dementia.

The insulin-like growth factor I (IGF-I) and its binding protein IGFBP-1 have been identified as biomarkers of insulin resistance and the risk of developing T2DM. Insulin-like growth factor-1 is mainly produced in the liver as a response to stimulus from growth hormone, but is also produced locally in different tissues in the body. It is a 70-amino acid polypeptide with a weight of 7.6 kDa and has six binding proteins that modulate the effects of IGF-1. It has anabolic effects in adults, with pleiotropic effects on both cell growth and metabolism. IGF-I is important for cell repair, survival, growth and metabolism.

It is well known that the IGF-1 levels will generally decrease with advancing age in humans. Thus, there is a clear clinical association between IGF-1 levels and age. Further, the concentrations of IGF-1 will decrease during inflammation, and in patients with ischemic heart disease, the amount of coronary calcium deposits has been reported to be inversely associated with the level of IGF-1. Further, in several studies, a low level of IGF-1 has been associated with an increased risk of ischemic heart disease or myocardial infarction, as seen in both human and animal models. Thus, there is a clear clinical association between a decrease in the concentration of IGF-1 and developing a disease as well as an association between a decrease in the concentration of IGF-1 and age.

IGFBP-1 has an IGF-dependent effect, transporting the IGF-I from the circulation to the target tissue where it, depending of its phosphorylation status, can both decrease and increase the binding of IGF-I to its receptor. IGFBP-1 also has an IGF-independent effect regulating the endothelial function via NO synthesis, cellular growth and migration via the binding to an integrin receptor as well as the synthesis of hepatic IGF-I. Both IGF-I and IGFBP-1 productions are regulated by insulin, nutrition and inflammation.

The IGF-binding protein-1 (IGFBP-1) is also known to be associated with cardiovascular diseases.

The main regulator of IGF-1 is growth hormone, and the main regulator of IGFBP-1 is insulin.

Measuring morning levels of IGFBP-1 will show the insulin produced during the previous 24 hours, thus a fasting level of IGFBP-1 can be used as a simple marker for insulin production.

It has been shown that low levels of IGFBP-1 can predict the future development of type 2 diabetes or the metabolic syndrome (obesity, hyperlipidaemia and hypertension) up to 16 years after the samples have been taken. In subjects with normal glucose tolerance, low levels of fasting IGFBP-1 indicate a several-fold increased risk of future T2DM, and high normal levels are shown to be a marker of decreased risk of developing T2DM even in subjects with family history of T2DM. The level of IGFBP-1 is a stronger predictor than BMI, waist circumference, fasting glucose and insulin levels. It has been shown that low levels are associated with hyperinsulinemia, insulin resistance and increased IGF-I activity. IGF-1 levels can be measured in the blood of humans in 10-1000 ng/ml amounts.

Further, it is generally believed that lifestyle interventions, such as diet control and physical activity, will increase the serum IGFBP-1 level, improve the glucose tolerance and reduce the risk of T2DM.

Coenzyme Q10, also sometimes referred to as Q10, CoQ10 or ubidecarenone, is produced in all tissues and cells in humans. This lipid, belonging to the group of neutral fat, has several established functions, such as mitochondrial respiration, energy production and antioxidants. Q10 influences several genes that regulate inflammation and cell death. A complete loss of Q10 will lead to cell death, and low concentrations have been found to be related to cardiomyopathy and a number of neuromuscular diseases. Further, it has been demonstrated that Q10 has positive effects on oxidative stress, inflammation and the immune defence system in subjects, both with and without diabetes. Positive protective effects on the renal function and peripheral nerve function have also been demonstrated in mice, and it is also believed that Q10 has positive effects on the endothelial function, increases the dilatation of the vascular system and decreases arterial stiffness. Studies in mice have shown that Q10 improves insulin sensitivity in the adipose tissue. Further, it has been shown that treatment with Q10 will decrease mortality in subjects with cardiac failure.

### SUMMARY

It has been demonstrated in clinical studies of elderly subjects that Q10 and selenium (from selenium yeast) supplements will improve insulin sensitivity and life expectancy. Reference is made to example 2 presented below.

In the clinical study mentioned in example 2 below, the participants had a mean age of 78 years. As the IGF concentration decreases with age, it was hypothesized that the placebo group would have a decrease in the IGF concentration over the study period. The aim of the study mentioned in Example 2 was to see if the participants randomized to receive treatment consisting of selenium and coenzyme Q10 would preserve their high IGF concentration compared to the placebo group. Thus, the aim of this study was to investigate a possible influence of selenium and coenzyme Q10 supplements for four years on IGF-1 and IGFBP-1 in an elderly healthy Swedish population.

The main finding from example 2 is that after treatment with selenium and coenzyme Q10 supplements, applying group mean evaluations, significantly higher IGF-1 and IGF-1 SD scores was seen in the active treatment group, whereas a decrease in concentration was seen of the same biomarkers in the placebo group.

Applying the repeated measures of variance evaluations, the same significant increase in concentrations of IGF-1 (F=68; P>0.0001), IGF-1 SD score (F=29; P<0.0001) and of IGFBP-1 (F=6.88; P=0.009) was seen, indicating the effect of selenium and coenzyme Q10 also on the expression of IGF-1 as one of the mechanistic effects of the intervention.

In conclusion, the results presented in example 2 show that selenium and coenzyme Q10 supplements over a four-year period result in increased levels of IGF-1 and the postprandial IGFBP-1, and an increase in the age-corrected IGF-1 SD score, compared with placebo in a Swedish elderly population.

From studies on an elderly healthy Swedish population, it has previously been reported that more than 90% had suboptimal selenium status. Thus, it was speculated that the positive results presented in example 2 below could well be the result of Q10 being the main factor responsible for the increased levels of IGF-1, whereas the selenium status could be of importance mainly—or exclusively—as an additional supplement for to subjects having inadequate selenium levels as a consequence of low-selenium diet.

These speculations led the present inventors to the preliminary hypothesis that Q10 is the main factor responsible for the increased levels of IGF-1, whereas the selenium status is hypothesised to be of much less importance, selenium mainly being of importance as an additional supplement for subjects having inadequate selenium levels as a consequence of low-selenium diet.

This hypothesis further suggests that the Q10 supplement in itself (i.e. without selenium supplementation) may be clinically relevant in preventing the development of T2DM. This lead to the hypothesis presented herein that treatment with Q10, especially when administered during 6 (12) months and given to subjects at high risk of developing T2DM, will show clinically relevant effect in reducing the risk of developing T2DM in a clinical placebo-controlled study.

In one aspect, the present disclosure relates to a method of treatment or prophylactic treatment of a human subject suffering from a disorder, wherein the human subject has serum levels of IGF-1 below 150 μg IGF-1/L serum, said treatment comprising administrating Q10 and optionally selenium to the subject. In some embodiments, the human subject has serum levels of IGF-1 below 148 μg IGF-1/L serum, such as below 146 μg IGF-1/L serum, below 144 μg IGF-1/L serum, below 142 μg IGF-1/L serum, below 140 μg IGF-1/L serum, or below 138 μg IGF-1/L serum.

In another aspect, the present disclosure relates to a method of treatment or prophylactic treatment of a human subject in need of higher serum levels of IGF-1, said treatment comprising administrating Q10 and optionally selenium to the subject. In some embodiments, the human subject has serum levels of IGF-1 below 150 μg IGF-1/L serum, such as below 148 μg IGF-1/L serum, such as below 146 μg IGF-1/L serum, below 144 μg IGF-1/L serum, below 142 μg IGF-1/L serum, below 140 μg IGF-1/L serum, or below 138 μg IGF-1/L serum.

In some embodiments, the human subject suffers from, or is prone to suffer from, a disorder resulting from a decrease in serum levels of IGF-1 to below 150 μg IGF-1/L serum. In a preferred embodiment the disorder is diabetes, preferably type 2 diabetes. In some embodiments, the human subject is selected from the group consisting of:

i. humans with decreased insulin sensitivity,
ii. humans with low IGFBP-1 fasting levels and/or low adiponectin fasting levels,
iii. humans above 40 years of age,
iv. women with a history of gestational diabetes, and
v. subjects with the metabolic syndrome.

In another aspect, the present disclosure relates to a method of attenuating a rate of decline of the weight ratio of serum levels of IGF-1 to IGFBP-1 in a human subject by administering Q10 and optionally selenium to the human subject.

In some embodiments, the rate of decline of the ratio of serum levels of IGF-1 to IGFBP-1 is attenuated to 0.5 per year or less, such as 0.48, 0.46, 0.44, 0.42, 0.40, 0.38, 0.36, 0.34, 0.32 or 0.30 per year or less. In a preferred embodiment, the human subject suffers from diabetes, preferably type 2 diabetes.

In another aspect, the present disclosure relates to a method of treating or preventing diabetes in a subject, comprising determining a weight ratio of serum levels of IGF-1 to IGFBP-1 in the subject, and administering an effective amount of Q10 and optionally selenium to the subject, if the weight ratio of serum levels of IGF-1 to IGFBP-1 is about 10 or lower, such as about 9.5, 9, 8.5, 8, 7.5 or 7 or lower.

In another aspect, the present disclosure relates to a method of treatment or prophylactic treatment of type 2 diabetes in a human subject being selected from the group consisting of:

i. humans with decreased insulin sensitivity,
ii. humans with low IGFBP-1 fasting levels and/or low adiponectin fasting levels,
iii. humans above 40 years of age,
iv. women with a history of gestational diabetes, and
v. subjects with the metabolic syndrome,

said treatment comprising administrating Q10 to the subject.

In some embodiments, the humans with decreased insulin sensitivity have increased HOMA-IR. In some embodiments, low adiponectin fasting levels are adiponectin serum levels of about 7.5 mg/l or less, such as 5 mg/l or less, or 2.5 mg/l or less. According to some embodiments, low IGFBP-1 fasting levels are IGFBP-1 serum levels of about 30 μg/I or less, such as about 25 μg or less, or 20 μg or less.

In one embodiment, the humans above 40 years of age have a family history of diabetes. According to another embodiment, the subjects with the metabolic syndrome suffer from hypertension, abdominal obesity, and/or high triglycerides.

According to another embodiment, the subjects with the metabolic syndrome do not suffer from diabetes mellitus type 2. According to another embodiment, the treatment or prophylactic treatment comprises the use of Q10 without simultaneous treatment with selenium supplementation. According to another embodiment, the treatment or prophylactic treatment comprises the use of Q10 in combination with a selenium compound.

According to another embodiment, the treatment or prophylactic treatment comprises the use of Q10 in combination with a selenium compound, with the proviso that the human subject has a daily intake of selenium resulting in a serum level of selenium of less than 90 μg/L, preferably less than 80 μg/L, even more preferably less than 70 μg/L.

In another embodiment, the Q10 is administered to a subject in a dosage regime comprising daily administration of 100 mg Q10 or more for at least 3 months.

In another embodiment, the Q10 is administered to a subject in a dosage regime comprising daily administration of 300 mg Q10 or more for at least 3 months, preferably at least 6 months, even more preferably 12 months.

In another embodiment, wherein the human subject has an age of 65 years or more.

In another embodiment, the Q10 is administered to a subject in a dosage regime comprising daily administration for at least 12 months, preferably at least 24 months, such as at least 36 months, or at least 48 months.

In another aspect, the present disclosure relates to a composition or kit-of-parts comprising Q10 and optionally selenium for use in the above-described methods.

Accordingly, the present disclosure also relates to Q10 for use in the treatment or prophylactic treatment of a human subject, said treatment comprising preventing a decrease in serum levels of IGF-1 and to the use of Q10 in the treatment or prophylactic treatment of a human subject, said treatment comprising preventing a decrease in serum levels of IGF-1.

More specifically, the present disclosure relates to Q10 for use in the treatment or prophylactic treatment of a human subject, said treatment comprising preventing a decrease in serum levels of IGF-1, preferably to below 150 μg IGF-1/L serum, and to the use of Q10 in the treatment or prophylactic treatment of a human subject, said treatment comprising preventing a decrease in serum levels of IGF-1 (preferably to below 150 μg IGF-1/L serum).

The present disclosure also relates to Q10 for use in the treatment or prophylactic treatment of type 2 diabetes in a human subject and to the use of Q10 in the treatment or prophylactic treatment of type 2 diabetes in a human subject.

More specifically, the present disclosure also relates to Q10 for use in the treatment or prophylactic treatment of type 2 diabetes in a human subject, said human subject having a high risk of developing T2DM, and to the use of Q10 in the treatment or prophylactic treatment of type 2 diabetes in a human subject, said human subject having a high risk of developing type 2 diabetes (T2DM).

Human subjects having a high risk of developing T2DM, as defined according to the present invention, are subjects selected from the group consisting of:
i. subjects with a decreased insulin sensitivity (increased HOMA-IR)
ii. subjects with low IGFBP-1 and adiponectin fasting levels
iii. subjects above 40 years of age with a family history of diabetes,
iv. female subjects with a history of gestational diabetes, and
v. subjects with a metabolic syndrome (hypertension, abdominal obesity, high triglycerides) but not having T2DM.

The Homeostasis Model Assessment (HOMA) estimates steady state beta cell function (% B) and insulin sensitivity (% S), as percentages of a normal reference population. HOMA IR (insulin resistance) is the reciprocal of % S (100/% S). According to the present invention increased HOMA-IR means a HOMA-IR level of at least 1.1, preferably at least 1.2 and even more preferably at least 1.3.

Low IGFBP-1 according to the present invention means a level of IGFBP-1 of 75% or less than the normal level of IGFBP-1, preferably a level of 70% or, even more preferably a level of 60% or less than the normal level of IGFBP-1. Normal serum levels of are estimated to be around 80.0 μg/l.

Low adiponectin fasting levels according to the present invention means a level of fasting adiponectin of 75% or less than the normal level of fasting adiponectin, preferably a level of 70% or, even more preferably a level of 60% or less than the normal level of fasting adiponectin. Normal serum levels of are estimated to be around 10.0 mg/l.

The present disclosure also relates to Q10 for use in the treatment or prophylactic treatment of type 2 diabetes in a human subject, said human subject having a high risk of developing T2DM, and said treatment comprising preventing a decrease in serum levels of IGF-1 to below 150 μg IGF-1/L serum, and to the use of Q10 in the treatment or prophylactic treatment of type 2 diabetes in a human subject, said human subject having a high risk of developing T2DM, and said treatment comprising preventing a decrease in serum levels of IGF-1 to below 150 μg IGF-1/L serum.

The present disclosure also relates to a method of treatment or prophylactic treatment of type 2 diabetes in a human subject having a high risk of developing T2DM as defined according to the present invention, said treatment comprising administrating Q10 to the subject.

Human subjects having a high risk of developing T2DM are selected from the group consisting of:
i. humans with a decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides), but not having T2DM.

### DETAILED DESCRIPTION OF THE INVENTION

IGF-1 levels can be measured in the blood of humans in 10-1000 ng/ml amounts. Serum levels of IGF-1 of 150 μg IGF-1/L serum or above are considered to be the normal level in middle-aged healthy adult human subjects not typically considered as having an increased risk of developing type 2 diabetes.

Thus, in a first aspect, the present invention relates to Q10 for use in the treatment or prophylactic treatment of a human subject, said treatment comprising the use of Q10 for preventing a decrease in serum levels of IGF-1. Preferably, the decrease in serum levels of IGF-1 according to the present invention means a decrease to below 150 μg IGF-1/L serum.

In a similar aspect, the invention relates to the use of Q10 for the treatment or prophylactic treatment of a human subject, said treatment comprising preventing a decrease in serum levels of IGF-1 below 150 μg IGF-1/L serum. In a similar aspect, the invention relates to the use of Q10 for the manufacture of a medicament for use in the treatment or prophylactic treatment of a human subject, said treatment comprising preventing a decrease in serum levels of IGF-1 to below 150 μg IGF-1/L serum.

In a second aspect, the present invention relates to Q10 for use in the treatment or prophylactic treatment of type 2 diabetes in a human subject, said human subject being selected from the group consisting of
i. humans with a decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM.

The homeostatic model assessment (HOMA-IR) is a method used to quantify insulin resistance and beta-cell function.

In a similar aspect, the present invention relates to the use of Q10 in the treatment or prophylactic treatment of type 2 diabetes in a human subject, said human subject being selected from the group consisting of:
i. humans with decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM.

In a similar aspect, the present invention relates to the use of Q10 for the manufacture of a medicament for the treatment or prophylactic treatment of type 2 diabetes in a human subject, said human subject being selected from the group consisting of:
i. humans with decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM.

In another aspect, the present invention relates to Q10 for use in the treatment or prophylactic treatment of type 2 diabetes in a human subject, said treatment comprising preventing a decrease in serum levels of IGF-1 to below 150 μg IGF-1/L serum, and said human subject being selected from the group consisting of:
i. humans with decreased insulin sensitivity (increased HOMA-IR)
ii. humans with low IGFBP-1 and adiponectin fasting levels
iii. humans being more than 40 years old (with a family history of diabetes),
iv. women with a history of gestational diabetes, and
v. subjects with the metabolic syndrome (hypertension, abdominal obesity, high triglycerides) but not T2DM.

In a similar aspect, the present invention relates to the use of Q10 in the treatment or prophylactic treatment of type 2 diabetes in a human subject, said treatment comprising preventing a decrease in serum levels of IGF-1 to below 150 μg IGF-1/L serum, and said human subject being selected from the group consisting of:
i. humans with decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM.

In a similar aspect, the present invention relates to the use of Q10 for the manufacture of a medicament for the treatment or prophylactic treatment of type 2 diabetes in a human subject, said treatment comprising preventing a decrease in serum levels of IGF-1 to below 150 μg IGF-1/L serum, and said human subject being selected from the group consisting of:
i. humans with decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM.

In all aspects of the present disclosure, prophylactic treatment is preferred.

### Dosages

In a preferred aspect of the present disclosure, Q10 is administered to the subject in a dosage regimen comprising daily administration of 100 mg Q10 or more for at least 3 months.

In a highly preferred aspect of the present disclosure, Q10 is administered to the subject in a dosage regimen comprising daily administration of 200 mg Q10 or more for at least 3 months.

In another highly preferred aspect of the present disclosure, Q10 is administered to the subject in a dosage regimen comprising daily administration of 300 mg Q10 or more for at least 3 months.

In another highly preferred aspect of the present disclosure, Q10 is administered to the subject in a dosage regimen comprising daily administration of 300 mg Q10 or more for at least 6 months.

Preferably, Q10 is administered to the subject in a dosage regimen for at least 6 months.

In another aspect, Q10 is administered to the subject in a dosage regimen for at least 12 months.

Preferably, Q10 is administered to the subject as 3 doses a day.

Preferably, Q10 is administered to the subject as 3 doses a day with 100 mg Q10 per dose.

In a highly preferred aspect of the present disclosure Q10 is in the crystal form of ubidecarenone, wherein the ratio of the crystal surface area measured in μm² per volume measured in μm³ of the ubidecarenone crystals is greater than 2 and wherein the crystal form of ubidecarenone comprises a plurality of substantially needle-shaped or cone-shaped elongated parts which have an average length of at least 10 μm and a diameter at the base of less than 1 μm, said elongated parts extending or protruding from a core region.

Preferred forms of Q10 are described in WO 2016/038146.

### Methods of Treatment

The present disclosure also relates to a method of treatment or prophylactic treatment of a human subject suffering from, or prone to suffer from, a decrease serum levels of IGF-1 to below 150 μg IGF-1/L serum, said treatment comprising administrating Q10 to the subject.

In another aspect, the present disclosure also relates to a method of treatment or prophylactic treatment of type 2 diabetes in a human subject, said treatment comprising administrating Q10 to the subject.

In a preferred aspect, the present disclosure relates to a method of treatment or prophylactic treatment in a human subject being selected from the group consisting of:
i. humans with decreased insulin sensitivity (increased HOMA-IR)
ii. humans with low IGFBP-1 and adiponectin fasting levels
iii. humans above 40 years of age (with a family history of diabetes),
iv. women with a history of gestational diabetes, and
v. subjects with the metabolic syndrome (hypertension, abdominal obesity, high triglycerides) but not T2DM, said treatment comprising administrating Q10 to the subject.
   Co-Treatment with a Selenium Compound

In general, the present disclosure is based on Q10 in itself (i.e. without selenium supplementation) being capable of providing adequate prevention of development of type 2 diabetes in a human subject.

Thus, in a preferred aspect, the present disclosure relates to Q10 for use in treatment or prophylactic treatment preventing the development of type 2 diabetes in a human subject, wherein the treatment or prophylactic treatment comprises the use of Q10 without simultaneous treatment with selenium supplementation.

However, it is generally known that certain geographical areas have a low basal selenium. Within populations inhabiting such areas, the daily intake of selenium may be suboptimal.

From previous studies on an elderly healthy Swedish population, it has been reported that more than 90% had suboptimal selenium status (PLoS One. 2016; 11(7):e0157541. doi: 10.1371/journal.pone.0157541. PubMed PMID: 27367855).

Especially in areas with low selenium content in the soil, or where residents have low daily intake of selenium, moderate selenium and coenzyme Q10 supplements will have positive effects, including effects on the levels of IGF-1 in the elderly, as part of the total clinical response.

Thus, as selenium to a certain extent seems to increase the effects of Q10 according to the invention, it is preferred in certain aspects of the invention to co-administer selenium with the Q10 administered according to the present disclosure.

Accordingly, and especially if the subject's diet contains inadequate amounts of selenium compounds, the treatment or prophylactic treatment according to the present disclosure comprises the use of Q10 in combination with a selenium compound.

Accordingly, in another aspect, the present disclosure relates to Q10 for use in treatment or prophylactic treatment preventing the development of type 2 diabetes in a human subject, wherein the treatment or prophylactic treatment comprises the use of Q10 in combination with a selenium compound, with the proviso that the human subject has a suboptimal daily intake of selenium.

A suboptimal intake of selenium is an intake of selenium-containing foods and supplements resulting in a serum level of selenium of less than 90 μg/L, preferably less than 80 μg/L, even more preferably less than 70 μg/L.

### Medical Kit

In one aspect, the present disclosure relates to a composition or kit-of-parts comprising Q10 and an instruction that Q10 is suitable for use in treatment or prophylactic treatment preventing the development of type 2 diabetes in a human subject.

Compositions according to the invention may be in the form of any suitable delivery vehicle, such as tablets, pills, capsules or other suitable vehicles comprising Q10. Preferably, the compositions or kit-op-parts according to the invention are accompanied by information regarding the intended use, and preferably additional information regarding instructions for use in the treatments described above.

In one aspect, the Q10 is comprised in suitable vehicles which do not contain additional nutritional supplements.

In a highly preferred aspect of the invention the composition comprising Q10 is a composition comprising ubidecarenone solubilized in an oil matrix, wherein the oil matrix comprises an oil solution consisting of approximately 4 parts oil per 1 part Q10 (w/w), wherein the oil consists of 75% soybean oil, 20% hydrogenated soybean oil with a melting point 32° C. and 5% hydrogenated soybean oil with a melting point 41° C. Preferred compositions are described in WO 2016/038150.

It should be understood that any feature and/or aspect discussed above in connection with the compounds according to the invention applies by analogy to the methods described herein.

The following examples are provided below to illustrate the present invention. They are intended to be illustrative and are not to be construed as limiting in any way.

### EXAMPLES

### Example 1: Planned Placebo-Controlled Clinical Study

### Patients and Methods

This study will be registered at Clinicaltrials.gov.

### Patient Eligibility

Subjects with a high risk of developing T2DM are exposed to intervention with Q10 supplement in a randomized placebo-controlled clinical trial for 6 months. The study comprises investigations of markers of insulin sensitivity and glucose tolerance.

Subjects with a high risk of developing T2DM are defined as:
i. subjects with decreased insulin sensitivity (increased HOMA-IR)
ii. subjects with low IGFBP-1 and adiponectin fasting levels
iii. subjects above 40 years of age with a family history of diabetes,
iv. female subjects with a history of gestational diabetes, and
v. subjects with a metabolic syndrome (hypertension, abdominal obesity, high triglycerides) but not T2DM.

Subjects 100 (50 men, 50 women) without diabetes but with low serum levels of fasting IGFBP-1 and adiponectin with a family history of T2DM or a history of gestational diabetes are asked if they will participate in the intervention study.

Prior to enrolment, candidate participants are screened for insulin resistance and for risk of developing T2DM by collecting morning fasting blood samples, anthropometric measurements, resting blood pressure and pulse, food and activity questionnaires. The blood will be tested for f-B-glucose, insulin, c-peptide, HbA1c, ApoB/ApoA1, triglycerides, HDLchol, LDLchol, total chol, ALT, AST, hsCRP, Hb, LpK, creatinine, IGFBP-1, IGF-I and adiponectin.

Candidate participants with low IGFBP-1 and adiponectin levels, f-B-glucose <6.0 mmol/L and HbA1c <44 mmol/mol are asked to come fasting (baseline visit) for an oral glucose tolerance test (OGTT) with determination of glucose and insulin at 0, 30, 60, 90 and 120 minutes and measures of endothelial function, urine samples and new blood tests before starting a placebo-controlled randomized clinical trial with per oral Q10 100 mg (Myoqinon, PharmaNord, Denmark) or placebo three times a day for 6 (12) months.

The intervention will be followed up after 3 and 6 (and 12) months of treatment compared to placebo with anthropometric measurements, resting blood pressure and pulse, food and activity questionnaires, blood and urine tests. OGTT and endothelial function tests will be repeated after 6 and (12) months.

Criterion for inclusion: 18-75 years of age, decreased insulin sensitivity (increased HOMA-IR), low IGFBP-1 and adiponectin fasting levels.

Criterion for exclusion: diabetes, CKD 4-5 (creatinine clearance <60 ml/min), pregnancy, cancer, chronic inflammatory disease, participation in other studies, myocardial infarction three months before the study.

### Study Design

After written informed consent, the persons are asked to come fasting in the morning for clinical examination and blood sampling for markers of endothelial function, oxidative stress, inflammation, metabolic glucose control, the IGF system, adiponectin levels and lipid profile. Urine samples are collected for analyses of F2-isoprostanes. The clinical examination includes endothelial function, resting blood pressure and pulse sitting, height, weight, waist and hip circumference, bio-impedance with determination of body fat distribution, food and activity questionnaires. This procedure will be repeated after 12 and 26 weeks. The study is a randomized placebo controlled study during 6 (12) months. At the baseline visit, the subject is given a closed envelope labelled A or B (placebo or Q10 Myoqinon (Pharma Nord, Denmark)) prescribing 100 mg three times a day during 26 weeks. An oral and written information on the importance of healthy diet (Mediterranean or Nordic) and physical activity is given. Blood is collected before treatment and after 12 and 26 weeks of treatment. Glucose tolerance tests (OGTTs) will be performed at baseline, 26 weeks. Microvascular endothelial function is performed at baseline, 26 weeks with ENDO-PAT 2000 (a non-invasive method), based on changes in the peripheral arterial pulse waves in the finger, basal and after 5 min of arterial occlusion during hyperaemia. Changes in pulse waves reflect endothelial function, and a decreased response has proved to be associated with insulin resistance.

The packages with Q10/placebo are returned to the clinic after 6 weeks when resting blood pressure and pulse sitting, weight, waist and hip circumference, bio-impedance with determination of body fat distribution are done and food and activity questionnaires are recorded. New packages of study compounds will be distributed. This procedure is repeated after 12 weeks (visit 3). The final visit is at 26 weeks (visit 4), when OGTT and endothelial function are repeated. The total blood volume taken is max 100 ml×3(4) during 26 (52) weeks.

### Example 2: Increase in Insulin-Like Growth Factor 1 (IGF-1) and Insulin-Like Growth Factor Binding Protein 1 after Selenium and Coenzyme Q10 Supplements

### Study Design

The present example is from a study aimed at examining the effects of selenium and coenzyme Q10 supplements on concentrations of IGF-1 and its binding protein IGFBP-1 in a population showing reduced cardiovascular mortality following such supplementation.

The design of the main study has been published in Int. J Cardiol. 2013; 167(5):1860-6. doi: 10.1016/j.ijcard.2012.04.156. PubMed PMID: 22626835.

In brief, 443 elderly healthy participants living in a rural municipality in the south of Sweden were randomized to dietary supplements of 200 mg/day of coenzyme Q10 capsules (Bio-Quinon 100 mg B.I.D, Pharma Nord, Vejle, Denmark) and 200 μg/day of organic selenium yeast tablets (SelenoPrecise 200 μg, Pharma Nord, Vejle, Denmark), or a similar placebo. Previous studies on an elderly healthy Swedish population have reported that more than 90% had suboptimal selenium status (PLoS One. 2016; 11(7):e0157541. doi: 10.1371/journal.pone.0157541. PubMed PMID: 27367855).

The study supplements were taken in addition to regular medication.

As the intervention time was unusually long (48 months), only 221 participants completed the study, 64 died during the total intervention time, and 129 (29.1%) decided not to complete the study. The reasons for the latter have been presented in detail in the main publication, but the main reason was that there were too many tablets to take. The first participant was included in January 2003, and the last participant concluded the study in February 2010.

Out of the total study population, 215 participants were analyzed regarding IGF-1, the age-corrected values of IGF-1 (IGF-1 SD) based on the standard deviation of the mean value based on 247 healthy individuals (see J Clin Endocrinol Metab. 1995; 80(9):2646-52. doi: 10.1210/jcem.80.9.7545695. PubMed PMID: 7545695 and Clin Endocrinol (Oxf). 2002; 57(6):793-803. PubMed PMID: 12460330), and the IGF-binding protein 1 (IGFBP-1).

Of the 215 participants, 117 had previously been randomized to active treatment (selenium and Q10 supplements) and 98 to placebo.

This study was registered at Clinicaltrials.gov and has the identifier NCT01443780.

A clinical examination was performed and blood samples were taken at the start of the study and after 48 months. Evaluations of IGF-1, the age adjusted IGF-1 SD score and IGFBP-1 were performed using group mean values, and repeated measures of variance.

### Biochemical Analyses

All blood samples were obtained while the patients were at rest in a supine position. The blood samples were collected in plastic vials containing EDTA (ethylenediamine tetracetic acid). The vials were placed on ice before chilled centrifugation at 3000 g, and then frozen at −70° C. No sample was thawed more than twice.

IGF-1 concentrations in plasma were determined by an in-house RIA after separation of the different IGFs from IGFBPs by acid ethanol extraction and cryoprecipitation. To minimize interference from the remaining IGFBPs, des (1-3) IGF-1 was used as a radioligand. The intra- and inter-assay coefficients of variation (CV) were 4% and 11%, respectively. Plasma concentrations of IGF-1 are age-dependent, decreasing with age, thus IGF-1 values are also expressed as standard scores (SD scores) calculated from the regression of the values of two populations of healthy adult subjects.

IGFBP-1 concentrations were determined by an in-house RIA using the method of Póvoa et al. (Acta Endocrinol (Copenh). 1984; 107(4):563-70. PubMed PMID: 6083690). The sensitivity of the RIA was 3 μg/L and the intra- and inter-assays CV were 3% and 10%, respectively.

### Statistical Methods

Descriptive data are presented as percentages or mean±standard deviation (SD). A student's unpaired two-sided t-test was used for continuous variables and the Chi-square test was used for analysis of one discrete variable. As the dataset demonstrated a slight non-Gaussian distribution, the dataset was transformed in order to obtain a normal distribution, which was controlled through Kolmogorov-Smirnov's test. Transformed data were used in the t-test evaluations as this evaluation is more sensitive to a non-normal distribution of data. As the ANOVA algorithm can handle a slight non-Gaussian distribution, non-transformed data were applied in the repeated measures of variance evaluation.

Evaluation of the effects of treatment were based both on group mean values, but also on use of a repeated measures of variance analysis where the values of the individual participant were identified during the two different measured time points. P-values <0.05 were considered significant, based on a two-sided evaluation. All data were analyzed using standard software (Statistica v. 13.2, Dell Inc, Tulsa, Okla.).

### Results

Of the study population, 113 were females, and 102 were males. In the population, about 71% had been diagnosed with hypertension, about 18% had ischemic heart disease, 17% had diabetes, and 5% had an impaired systolic cardiac function defined as EF<40%. The different covariates were well-balanced between the active versus the placebo group, and it could also be seen that the subgroup analyzed was representative of the original study population consisting of 443 individuals.

At the start of the intervention, the serum levels of IGF-1 in the subgroups of those given selenium and coenzyme Q10 combined were not significantly different from values in those given the placebo (t=1.82; P=0.06). However, at the end of the intervention period, a significantly higher serum level of IGF-1 could be seen in the active treatment group compared with the placebo group (183 vs. 166 microgram/L; t=5.78; P<0.0001). A significant increase in the serum IGF-1 was observed in the active treatment group (from 154 to 183 microgram/L; t=4.38; P<0.0001), whereas a decrease was seen in the placebo group (166 vs. 144 microgram/L; t=3.43; P=0.0007).

However, as group mean values do not necessarily express the individual change of levels, repeated measures of variance were also performed. Applying repeated measures of variance, using group (active and placebo) and follow-up (baseline and 48 months), a significant effect on serum levels of IGF-1 (i.e. group*follow-up interaction), F=68; P<0.0001 could be seen, favouring those receiving selenium and coenzyme Q10. Thus, an increase in IGF-1 concentration in those receiving the active substance and a decrease in those given the placebo could be seen.

Applying an age-corrected score of IGF-1, the IGF-1 SD, the same result was obtained; at the start of the intervention, no statistical difference in score between the active treatment group and the placebo group could be seen (t=0.06; P=0.96). However, at the end of the intervention, a highly significant difference could be noted between the groups, with a higher score in the active treatment group (t=3.11; P=0.002).

In the active treatment group, a tendency to increase in score was noted during the intervention (from 1.22 to 1.58; P=0.05), whereas a non-significant decrease was noted in the placebo group (from 1.26 to 0.95; P=0.16). Applying the repeated measure of variance methodology, as reported above, a highly significant effect on IGF-1 SD could be demonstrated (F=29; P<0.0001).

In order to evaluate the influence of the basal serum selenium concentration on the response to selenium and coenzyme Q10 supplements as seen in the concentration of IGF-1, we performed evaluations of the subgroup having the lowest quartile of basal selenium concentration, and compared the response of IGF-1 with those having the highest quartile. It could be seen that the response to selenium supplement on IGF-1 concentration was significantly higher in those where there was a selenium deficiency compared with those with no, or little deficiency.

### Estimated Active Form of IGF-1

In literature, the use of the ratio of IGF-1/IGFBP-1 as a measure of the active form of IGF-1 has been proposed. A decline in this ratio could be reported in the placebo group during the intervention time (9.72 to 6.31; t=2.37; P=0.019), whereas in the active treatment group, a trend towards a decline during the intervention time could be seen (10.13 to 8.97; t=2.00; P=0.05), and comparing the ratios of IGF-1/IGFBP-1 between the active and the placebo groups after the intervention a significant difference could be reported (8.97 versus 6.31; t=2.18; P=0.04).

IGF-Binding Protein-1 and Intervention with Selenium and Coenzyme Q10

The plasma concentrations of one of the six IGF-binding proteins, IGFBP-1, were determined 2-3 hours after a meal. At the start of the intervention, no statistical difference in mean concentrations between the active treatment group, and the placebo group could be noted (30 vs. 32 microgram/L; P=0.25). At the end of the intervention, no significant difference could be noted (44 vs. 37 microgram/L; P=0.45). However, applying the repeated measure of variance methodology, a significant difference could be demonstrated (F=6.88; P=0.009), with a higher increase in the IGFBP-1 concentration in the active group compared with the active treatment group.

Further, the participants having a concentration of IGFBP-1 in the highest quartile (Q4) were examined and compared to the number in the active treatment group with that of the placebo group. No difference in the numbers of participants in Q4 could be seen at the start of the intervention (24/98 versus 29/117: P=0.96). However, after the intervention, compared to the placebo group, a significantly higher number of participants could be seen in Q4 of IGFBP-1 from the active treatment group (16/98 versus 36/117; X2=6.07; P=0.014).

### Summary of Results

In the present example, raised concentrations of IGF-1 and IGF-binding protein-1 in the participants receiving the active intervention, upon the follow-up investigation of the population at the end of the study period was demonstrated. In contrast, decreasing concentrations in those receiving the placebo was demonstrated, which was as expected in this group of aged Swedish individual.

As the intervention time was unusually long, four years, and the study population consisted of elderly individuals with a relatively low basal selenium status, we hypothesized that there would be a decrease in the levels of IGF-1 in the placebo group and preserved levels in those given active supplements.

However, upon relating IGF-1, IGF-1 SD and IGFBP-1 levels at baseline to the basal levels of selenium, no significant correlations were seen. Here, it should be noted that the present population was relatively homogenous with respect to selenium status, with median serum selenium of 67.1 μg/L, all individual values being below a proposed optimum of 90 μg/L.

When expressing the free and active fraction of IGF-1 as the ratio IGF-1/IGFBP-1, we found that the intervention caused a significantly attenuated decline in the active form of IGF-1.

The higher IGFBP-1 levels seen in those treated with Q10 and selenium suggest a decreased insulin response to the meal due to improved insulin sensitivity.

These results indicating increased insulin sensitivity are interesting in relation to previous concerns about the possibility that selenium supplement should cause an increased risk of diabetes (Environ Health Perspect. 2009; 117(9):1409-13. doi: 10.1289/ehp.0900704. PubMed PMID: 19750106; PubMed Central PMCID: PMCPMC2737018).

1. A method of treatment or prophylactic treatment of a human subject suffering from a disorder, wherein the human subject has serum levels of IGF-1 below 150 μg IGF-1/L serum, said treatment comprising administrating Q10 and optionally selenium to the subject.

2. A method of treatment or prophylactic treatment according to claim 1, wherein the human subject suffers from, or is prone to suffer from, a disorder resulting from a decrease in serum levels of IGF-1 to below 150 μg IGF-1/L serum.

3. A method of treatment or prophylactic treatment according to claim 1, wherein the disorder is diabetes, preferably type 2 diabetes.

4. A method of attenuating a rate of decline of the weight ratio of serum levels of IGF-1 to IGFBP-1 in a human subject by administering Q10 and optionally selenium to the human subject.

5. A method according to claim 4, wherein the rate of decline of the ratio of serum levels of IGF-1 to IGFBP-1 is attenuated to 0.5 per year or less.

6. A method according to claim 4, wherein the human subject suffers from diabetes, preferably type 2 diabetes.

7. A method of treating or preventing diabetes in a subject, comprising:
determining a weight ratio of serum levels of IGF-1 to IGFBP-1 in the subject, and
administering an effective amount of Q10 and optionally selenium to the subject, if the weight ratio of serum levels of IGF-1 to IGFBP-1 is about 10 or lower.

8. A method of treatment or prophylactic treatment of type 2 diabetes in a human subject being selected from the group consisting of:
i. humans with decreased insulin sensitivity,
ii. humans with low IGFBP-1 fasting levels and/or low adiponectin fasting levels,
iii. humans above 40 years of age,
iv. women with a history of gestational diabetes, and
v. subjects with the metabolic syndrome,
said treatment comprising administrating Q10 to the subject.

9. A method of treatment or prophylactic treatment according to claim 8, wherein the humans with decreased insulin sensitivity have increased HOMA-IR.

10. A method of treatment or prophylactic treatment according to claim 8, wherein low adiponectin fasting levels are adiponectin serum levels of about 7.5 mg/l or less, such as 5 mg/l or less, or 2.5 mg/l or less.

11. A method of treatment or prophylactic treatment according to claim 8, wherein low IGFBP-1 fasting levels are IGFBP-1 serum levels of about 30 μg/l or less, such as about 25 μg or less, or 20 μg or less.

12. A method of treatment or prophylactic treatment according to claim 8, wherein the humans above 40 years of age have a family history of diabetes.

13. A method of treatment or prophylactic treatment according to claim 8, wherein the subjects with the metabolic syndrome suffer from hypertension, abdominal obesity, and/or high triglycerides.

14. A method of treatment or prophylactic treatment according to claim 8, wherein the subjects with the metabolic syndrome do not suffer from diabetes mellitus type 2.

15. A method of treatment or prophylactic treatment according to claim 8, wherein the treatment or prophylactic treatment comprises the use of Q10 without simultaneous treatment with selenium supplementation.

16. A method of treatment or prophylactic treatment according to claim 8, wherein the treatment or prophylactic treatment comprises the use of Q10 in combination with a selenium compound.

17. A method of treatment or prophylactic treatment according to claim 8, wherein the treatment or prophylactic treatment comprises the use of Q10 in combination with a selenium compound, with the proviso that the human subject has a daily intake of selenium resulting in a serum level of selenium of less than 90 μg/L, preferably less than 80 μg/L, even more preferably less than 70 μg/L.

18. A method of treatment or prophylactic treatment according to claim 8, wherein the Q10 is administered to a subject in a dosage regime comprising daily administration of 100 mg Q10 or more for at least 3 months.

19. A method of treatment or prophylactic treatment according to claim 8, wherein the Q10 is administered to a subject in a dosage regime comprising daily administration of 300 mg Q10 or more for at least 3 months, preferably at least 6 months, even more preferably 12 months.

20. A method of treatment or prophylactic treatment according to claim 8, wherein the human subject has an age of 65 years or more.

21. A method of treatment or prophylactic treatment according to claim 8, wherein the Q10 is administered to a subject in a dosage regime comprising daily administration for at least 12 months, preferably at least 24 months.

22. Composition or kit-of-parts comprising Q10 for use in a method according to claim 1.
